(19) United States
(12) Patent Application Publication
(10) Pub. No.: US 2008/0125715 A1
(43) Pub. Date: May 29, 2008

(54) Shaped Introducer For Vascular Access

(76) Inventor: David Elliot Cohen, Wyckoff, NJ (US)

Correspondence Address: SILLS CUMMIS EPSTEIN & GROSS P.C., ONE RIVERFRONT PLAZA, IP DEPARTMENT, NEWARK, NJ, 07102, US

(21) Appl. No.: 11/942,635

(22) Filed: Nov. 19, 2007

Related U.S. Application Data
(60) Provisional application No. 60/860,678, filed on Nov. 21, 2006.

(51) Int. Cl. A61M 25/09; A61M 25/01; A61M 25/16; B65D 69/00

(52) U.S. Cl. 604/164.13; 604/164.01; 604/167.05; 206/363

(57) ABSTRACT

A device and method of providing vascular intervention is provided. The method involves the steps of inserting a guidewire having a flexible tip into an iliac artery and into an aorta, inserting a sheath having an introducer therethrough, the introducer having an angulated flexible tip, into the iliac artery over the guidewire, wherein the angulated tip of the introducer is also inserted into the aorta, and pulling back the guidewire from the introducer so that the angulated flexible tip of the introducer falls over an aortic bifurcation into a contralateral iliac artery. A kit is also provided, the kit having a sheath, an introducer running through the sheath and extending past a distal end of the sheath, and a guidewire running through the sheath and introducer; the guidewire having a flexible tip at a distal end and a stiff portion proximal to the flexible tip.

This application claims the benefit of provisional patent application Application No. 60/860,678, filed on Nov. 21, 2006.

### BACKGROUND OF THE INVENTION

1. Field of the Invention

The present invention relates to medical instruments and particularly to a shaped, tapered introducer within a sheath and which is utilized in vascular interventions that require arterial catheter access from a femoral artery to the contralateral leg.

2. Prior Art

One of the major causes of morbidity and mortality in modern times is the frequent occurrence of vascular disease. This disease expresses itself in heart attacks, strokes, lower extremity occlusive disease, and aneurysmal disease. The predominant cause of the vascular disease is atherosclerosis, which arises from localized plaque accumulation at various sites in the vascular system, and particularly in the arterial network of a person. Plaque accumulation is caused by deposition of fat, lipid, and the like substances, on the interior walls of blood vessel, which, over time, leads to narrowing of the vascular lumen, a phenomenon medically known as “stenosis”. As a result, blood flow is restricted and the person's health may be at serious risk, depending upon various other clinical and health-related factors.

Although atherosclerosis is commonly associated with coronary arteries that feed the heart, other peripheral blood vessels, such as carotid and lower extremity arteries are equally susceptible to plaque formation. As noted above, atherosclerosis is caused by localized plaque build up at certain vulnerable sites in the vascular system. In this regard, the coronary arteries, carotid bifurcation, infrarenal abdominal aorta, and iliofemoral vessels are particularly susceptible, while the thoracic aorta, common and distal internal carotid, mesenteric, intercostal, mammary, and upper extremity arteries tend not to be severely affected.

There are numerous techniques and devices used to open blocked arteries. One of the widely used and accepted techniques is known as “balloon angioplasty”. This technique involves introducing a catheter through a blood vessel to access the site of plaque formation. The catheter at its tip includes a balloon which is selectively inflated at the site to push or flatten the plaque against the walls of the blood vessels. This results in dilation (or dilatation) of the vessel lumen thereby improving the blood flow. A stent is commonly positioned subsequent to dilatation to prevent plaque regression and for maintaining the integrity of the vessel. Other approaches to dilatation of the blood vessels include using a cutting tool to remove the plaque (atherectomy) and using a laser to vaporize portions of the plaque. Often one or more medications are also infused through catheters to dissolve clots in arteries.

A key issue in the successful completion of a complex endovascular intervention is obtaining vascular access to the blockage. Two standard approaches are available for femoral, popliteal and tibial artery interventions in the leg: (1) the contralateral crossover and (2) the ipsilateral antegrade approaches. The crossover approach using a retrograde access of the common femoral artery (CFA) is the most commonly used access technique for infra-inguinal interventions. As compared to the antegrade approach, the crossover approach is technically easier and is associated with a lower complication rate. An important advantage of the contralateral approach is that management of the access site is remote from the treatment site. Thus, the post-interventional flow in the treated artery will not be affected by the routine post-procedural compression of the access site, which reduces the risk of post-interventional thrombosis in the treated artery. Finally, the crossover approach allows the treatment of very proximal femoral lesions and ipsilateral iliac artery lesions that would not be accessible by the antegrade approach.

In order to provide a conduit for interventional devices and to allow for contrast injections to guide the physician, the insertion of a crossover sheath is necessary. After an arterial sheath has been placed in the CFA in a standard retrograde fashion, the contralateral iliofemoral system is reached by placing a 5 French or 6 French angulated diagnostic catheter (e.g. Cobra, Hook, Shepherd-Crook, IMA, Simmons, omniflush, diagnostic right coronary catheter) at the aortic bifurcation. The catheter is manipulated so that the tip of the catheter “engages” the ostium of the contra-lateral common iliac artery. Typically, a soft, hydrophilic guidewire (0.035″) is navigated through the guide catheter and advanced down the contralateral iliac artery and into the femoral artery and the diagnostic catheter is then advanced over this wire into the iliac artery. The soft, 0.035″ guidewire is then exchanged for a stiff guidewire (e.g. Terumo 0.035″ stiff angled glide wire, 0.035″ Guidant supra core wire, Amplatz, ultrastiff, Boston Scientific). The stiff guidewire opens the angle of the aortic bifurcation and facilitates the placement of the appropriate crossover sheath. A crossover sheath, (e.g. Cook 6-8 French, 45-90 cm) is advanced over the stiff guidewire and into the contralateral common femoral artery or superficial femoral artery.

Currently available sheaths are typically straight and are provided with a tapered introducer within the sheath. The sheath, with its introducer inside, is typically advanced into the artery at which time the “introducer” is removed. At that time, a separate angulated catheter is inserted into the sheath and this is used to perform an aortogram followed by the guiding of a wire into the opposite leg. Once the wire is properly positioned, the catheter and sheath are advanced over the aortic bifurcation. Frequently the sheath and catheter do not function smoothly together because of differences in the diameters of the two (creating undesirable transitions) or because of improper stiffness. Oftentimes the sheath's introducer is too stiff to cross over the bend. As such, this often requires trying a variety of catheter/sheath/introducer/wire combinations in order to advance the sheath successfully over the bifurcation. The lack of a unified, dedicated sheath-introducer system for this maneuver often results in prolonged effort, valuable procedural time, multiple catheters and wires, and risk of complications.

One currently available sheath is the “Balkan Crossover Sheath” by Cook which is designed for vascular interventions and aortography procedures and which includes a bend into the sheath, but which uses a stiff, short, straight introducer. This “Balkan” sheath has a natural bend in it, but the straight, unshaped, and stiff introducer is not amenable to direct advancement over the bifurcation. Cook also manufactures the Shuttle Select Sheath which is designed only for access into the carotid artery only.

### SUMMARY OF THE INVENTION

The device of the present invention includes an integrated sheath/introducer combination designed to function together for easily advancing the sheath over the aortic bifurcation. The sheath has a valve-like hub in its proximal end and a “sidearm” with a stopcock to permit injections. Inside the sheath is an inner angulated “introducer”. The angulated introducer differs from existing sheath devices in several important ways:

1. The introducer will have a highly angulated tip to allow easy advancement of the wire into the contralateral iliac artery.

2. The introducer will be significantly longer (protruding approximately 8-10 cm beyond the sheath tip), more flexible, more hydrophilic, and more highly tapered than existing introducers. The introducer will taper up in diameter to match the sheath diameter exactly (making for a transitionless junction of introducer and sheath), and which is made of hydrophilic materials.

3. The introducer would also have “sideholes” near the angulated tip to permit improved quality angiograms without needing to place a diagnostic catheter through the sheath for this purpose.

The integrated introducer of the present invention will be longer, more tapered and flexible than existing products, and will have a distal curved or angulated shape built into it. The introducer will have a non-kinking, braided sheath design and made from hydrophilic surfaces to facilitate advancement. The introducer will have diameters of 5 French, 6 French, 7 French, 8 French, and 9 French with sheath lengths of 45 cm, 65 cm and 90 cm.

The design of the introducer of the present invention would allow for aortography (dye injection) to be done through the introducer, “hook” the bifurcation with the introducer, advance the guidewire over the bifurcation, and then use the extra long, highly flexible and hydrophilic introducer to advance sufficiently into the opposite iliac artery to allow one-step advancement of the sheath over the bifurcation.

Accordingly, a method of providing vascular access is provided, the method comprising inserting a guidewire having a flexible tip into an iliac artery and into an aorta, inserting a sheath having an introducer therethrough, the introducer having an angulated flexible tip, into the iliac artery over the guidewire, wherein the angulated tip of the introducer is also inserted into the aorta, and pulling back the guidewire from the introducer so that the angulated flexible tip of the introducer falls over an aortic bifurcation into a contralateral iliac artery.

The method further comprises injecting dye through the sheath through sideholes on the flexible tip of the introducer to allow vessels to be visualized through x-rays. The method further comprises re-inserting the guidewire so that it travels through the sheath and introducer into the contralateral iliac artery, and advancing the sheath and introducer over the guidewire over the aortic bifurcation and into the contralateral iliac artery, and removing the guidewire and introducer from the sheath.

The flexible tip of the guidewire can be approximately 10 cm in length and the guidewire can be stiff proximate to the tip of the guidewire. The introducer can be tapered, and the diameter of the introducer and sheath is such that the introducer fits snugly into the sheath to provide a transitionless seam of the introducer from the sheath.

Further, a device for vascular access is provided, the device comprising a sheath having a distal end and a proximal end, and an introducer running through the sheath and extending past a distal end of the sheath, the introducer having a flexible angulated tip at a distal end of the introducer, wherein the introducer fits snugly into the sheath to provide a transitionless extension of the introducer from the sheath.

The device for vascular access further comprises a valve-like hub at a proximal end of the sheath, and a stopcock and sidearm at a proximal end of the sheath for injecting fluid or dye through the sheath and for measuring pressure. A plurality of sideholes can be provided on the flexible angulated tip of the introducer.

The introducer can be tapered, having a larger diameter at its proximal end and a smaller diameter at its tip. The introducer can be hydrophilic. The flexibility of the transducer can vary from more flexible at its tip to less flexible more proximally. The introducer can protrude approximately 8-10 cm beyond the distal end of the sheath. The introducer can be made of a continuous metal or polymeric coil. The sheath can range in length between 45 cm to 90 cm.

Finally, a kit is provided for vascular access, the kit comprising a sheath having a distal end and a proximal end, an introducer running through the sheath and extending past a distal end of the sheath, the introducer having a flexible angulated tip at a distal end of the introducer, and a guidewire running through the sheath and introducer; the guidewire having a flexible tip at a distal end.

The guidewire can be stiff proximal to the flexible tip. The stiffness of the wire can correlate with a stiffness of the introducer. The flexible tip of the guidewire can run a length of approximately 10 cm. The guidewire can have an angulated flexible tip.

The above and other features of the invention, including various novel details of construction and combinations of parts, will now be more particularly described with reference to the accompanying drawings and pointed out in the claims. It will be understood that the particular device embodying the invention is shown by way of illustration only and not as a limitation of the invention. The principles and features of this invention may be employed in various and numerous embodiments without departing from the scope of the invention.

### BRIEF DESCRIPTION OF THE DRAWINGS

These and other features, aspects, and advantages of the apparatus and methods of the present invention will become better understood with regard to the following description, appended claims, and accompanying drawings where:

FIG. 1 illustrates a side see-through view of a prior art arterial sheath and introducer;

FIG. 2 illustrates a side see-through view of one embodiment in accordance with the present invention; and

FIGS. 3a-3e illustrate the technique of contralateral access to the iliac artery for interventional procedures using the embodiment illustrated in FIG. 2 in accordance with the present invention.

### DETAILED DESCRIPTION OF THE INVENTION

Although this invention is applicable to numerous and various types of vascular access, it has been found particularly useful in the field of vascular interventions that require arterial catheter access from a femoral artery to the contralateral leg. Therefore, without limiting the applicability of the invention to the above, the invention will be described in such environment.

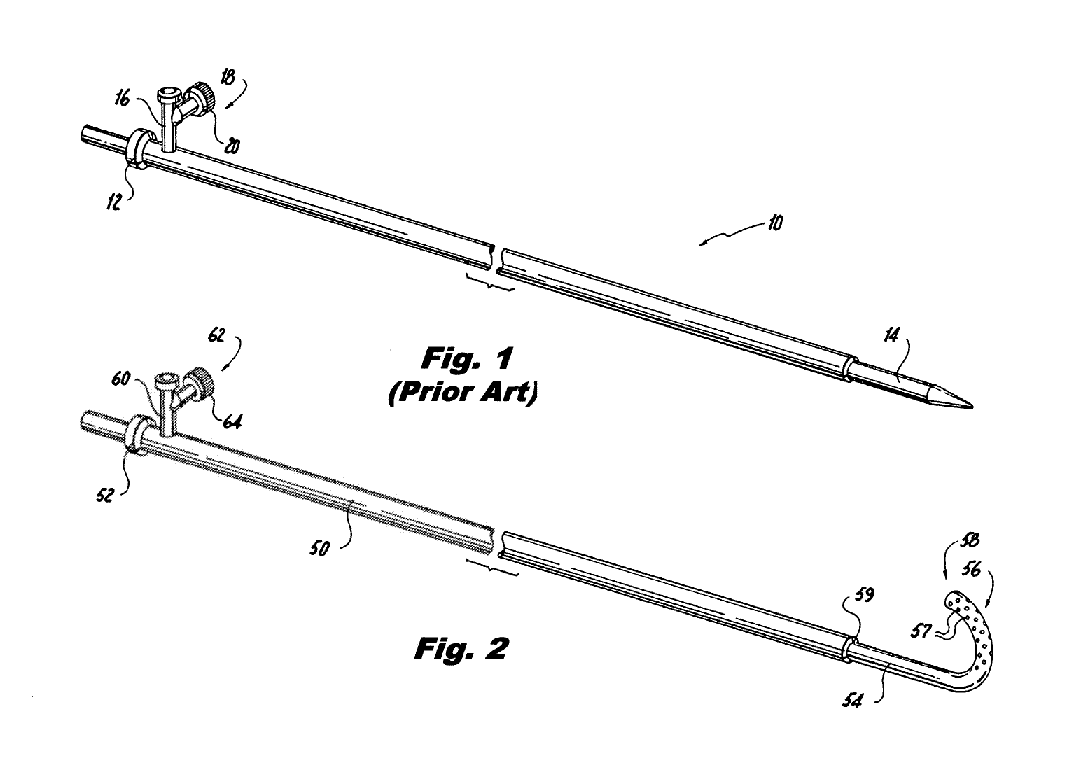

FIG. 1 illustrates a prior art arterial sheath 10 having a valve-like hub 12 at one end and containing a co-axial introducer 14 which protrudes from an opposite end of sheath 10. A sidearm 16 is connected to the sheath 10 at a proximal end 18 of sheath 10, and has a stopcock 20 connected with the sidearm 16 and which facilitates injections through sheath 10.

The present invention is illustrated in FIG. 2 which shows a sheath 50 having a valve-like hub 52 at one end and a modified introducer 54 at the opposite end. Introducer 54 fits snuggly within and through sheath 10 to provide a transitionless extension of introducer 54 from sheath 50. The introducer 54 will be significantly longer (protruding approximately 8-10 cm beyond the sheath tip 59), more flexible, more hydrophilic, and more highly tapered than existing introducers. The introducer 54 will taper up in diameter to match the sheath 50 diameter exactly (making for a transitionless junction of introducer and sheath), and which is made of hydrophilic materials.

The tip 56 of the introducer will be highly angulated and have a plurality of sideholes 56 about a distal end 58 of introducer 54. A sidearm 60 is connected to sheath 50 at a proximal end 62 of sheath 50, and has a stopcock 64 connected with sidearm 60 which allows for injection of dye or fluid through sheath 50.

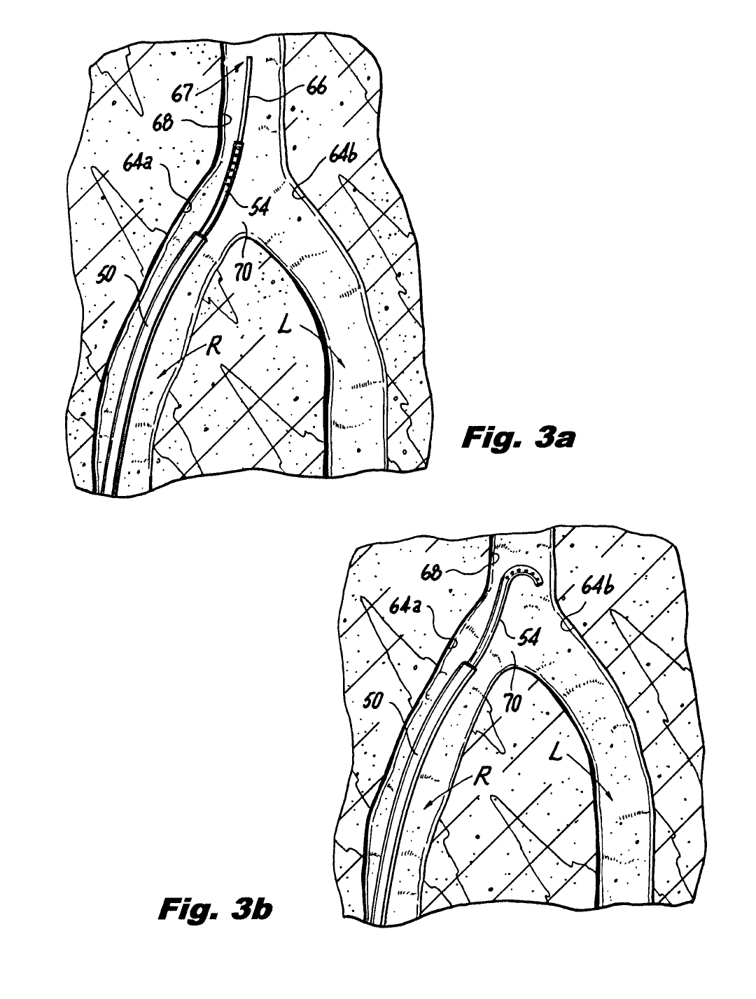

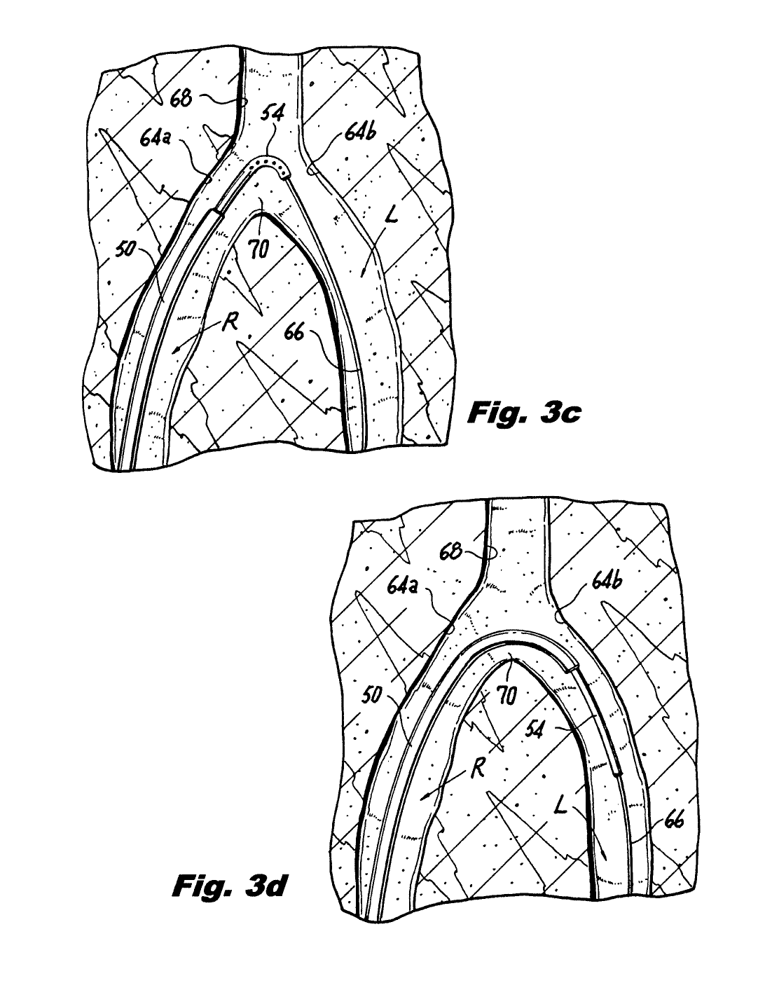

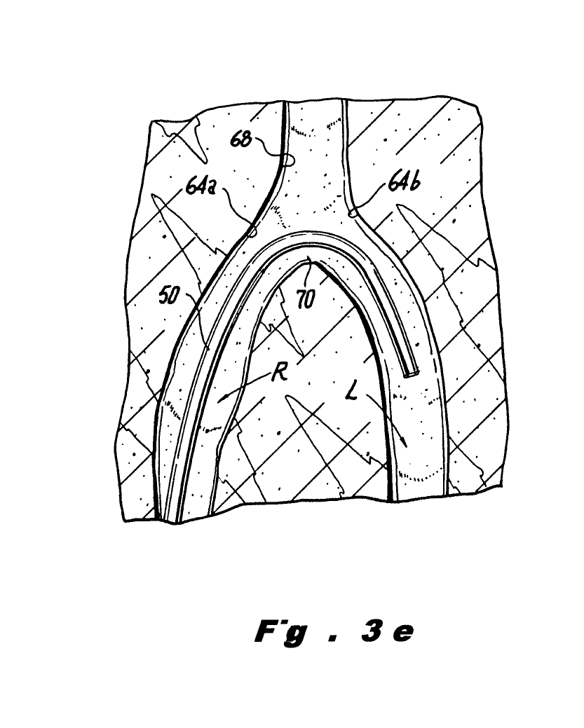

Now, with reference to FIGS. 3a-3e, a method of using the device of the present invention will be described. FIGS. 3a-3e illustrate the technique of insertion of sheath 50 into a patient's iliac artery 64a and subsequently into the contralateral iliac artery 64b. For simplicity and the sake of illustration only, in FIG. 3a, sheath 50 is inserted in the iliac artery 64a from the patient's right side (indicated in FIGS. 3a-3e by “R”). Sheaths typically follow a guidewire (such as wire 66) which had been introduced through a hollow needle during procedures such as the Seldinger technique which is described in S. I. Seldinger, Acta Radiol. 1953 May; 39(5):368-76, and which is incorporated by reference herein in its entirety.

In FIG. 3a, a guidewire 66 having a tip 67 at a distal end is advanced from the needle puncture site (not shown) in the right femoral artery. The tip 67 of the guidewire 66 is inserted first. The tip 67 of the guidewire 66 is flexible at a distal end for an approximate length of 10 cm, and stiffer or firmer after 10 cm, or proximal to the tip 67. The guidewire 66 is inserted until at least the tip 67 is extended into the aorta 68. Then, sheath 50 and introducer 54 are inserted over the guidewire 66 until at least the tip 56 of the introducer reaches into the aorta 68.

With the guidewire 66 inserted first into the patient, introducer 54, which is made of a flexible, non-kinking, hydrophilic material, assumes a “straight” shape so as to follow the straight guidewire 66. After the sheath 50 and introducer 54 are inserted, guidewire 66 is pulled back (FIG. 3b), allowing the tip 56 of the curved introducer 54 (now in the aorta 68) to assume its normal curve pointing downward towards the other (left) iliac artery 64b (indicated in FIGS. 3a-3e by “L”). Sideholes 57 in introducer 54 facilitate the angiogram procedure by allowing a contrast material, typically a radiopaque substance to flow through sheath 50 and introducer 54 and into the blood vessels to allow the vessels to be visualized via X-rays (angiography). Dye can now be injected through the introducer allowing for visualization of the arteries.

Guidewire 66 is then re-inserted so that it will now travel over the aortic bifurcation 70 and down the left iliac artery 64b as illustrated in FIG. 3c. This is where it is important for the tip 67 of the guidewire 66 to be flexible. The flexibility of the tip 67 of the guidewire 66 allows the tip 67 of the guidewire 66 to follow the introducer 54 into the left iliac artery 64b. Otherwise, if the tip 67 of the guidewire 66 were firm, the introducer 54 would straighten as the guidewire 66 was re-inserted and the guidewire 66 and introducer 54 would proceed into the aorta 68. The flexibility of the tip 67 of the guidewire 66 allows the guidewire 66 to be re-inserted into the left iliac artery 64b.

Sheath 50 (following introducer 54) is then advanced over the aortic bifurcation 70 into the other iliac artery 64b as illustrated in the FIGS. 3c-3d. As shown in FIG. 3e, the guidewire 66 and introducer 54 are then removed from the sheath 50 which can now be used to inject dye or deliver treatment to the artery beyond it.

Introducer 54 is typically a guiding introducer containing a lumen running lengthwise therethrough and having a proximal end 50a and distal end 50b. Introducer 54 plays an essential role in providing a facilitating pathway for a treatment or passage of a therapeutic catheter (not shown).

Referring more particularly to the construction of introducer 54 of the sheath 50, it will have a reinforced characteristic so as to exhibit adequate torsional responsiveness and column strength in order to suitably perform as a guiding catheter, as well as a sheath introducer. Elimination or minimization of kinking is also an important objective. Typically, these reinforcing functions are accomplished by the inclusion of braiding, such as of a continuous metal or polymeric coil positioned within or embedded within the walls of the guiding sheath. Also, selection of materials for the walls of the tubular body, including polymer coextrusion approaches, which include relatively stiff extrusion layer(s), can also address the need for this reinforced attribute. Sheath 50 in a preferred embodiment of the present invention is integral and coaxial with introducer 54.

The typical sheath 50 in accordance with the present invention will be available as 5 French, 6 French, 7 French, 8 French and 9 French sizes with lengths of 45 cm, 65 cm and 90 cm, but is not limited to these sizes.

The sidearm 60 and stopcock 64 are used to inject fluids or dye and to measure pressures.

Introducer 54 will protrude approximately 8-10 centimeters beyond the distal tip 59 of sheath 50. Introducer 54 will be tapered so that its junction with sheath 50 will be transitionless and its junction with wire 66 protruding from its tip will likewise be transitionless. Introducer 54 is advantageously hydrophilic. The flexibility of introducer 54 will transition from very flexible at its tip, and less flexible more proximally. In a preferred embodiment, the stiffness and flexibility of the introducer 54 will correlate to the stiffness and flexibility of the guidewire 66.

The guidewire 66 can be a hydrophilic wire with an angled tip, and can range in length from 100 cm to 300 cm, but is preferably approximately 190 cm in length. The distal end 67 (end that first enters the iliac artery) is preferably highly soft and flexible for a length of approximately 10 cm. The flexibility allows the advantages described above and also is non-traumatic to the patient. Proximal to those 10 cm, the guidewire 66 will be extremely stiff.

This guidewire configuration will expedite the following functions:

1. The flexible tip will allow for atraumatic entry into the femoral artery and negotiation of the iliac artery into the aorta.

2. The flexible tip will allow for easy contralateral wire access without prolapsing the sheath introducer tip up into the aorta.

3. The stiff rear-end of the guidewire will facilitate direct insertion of the soft, angulated sheath introducer tip into the femoral artery through the skin. It will obviate the need to use a stiffer introducer to pass through the skin and enter the femoral artery.

4. The stiff rear-end of the guidewire will also facilitate advancement of the sheath/introducer assembly over the aortic bifurcation. Once the flexible tip of the guidewire is advanced over the bifurcation, it is advanced further allowing the stiffer portion of the guidewire to pass over the bifurcation, thereby facilitating advancement of the sheath over the bifurcation.

5. This configuration will help to avoid needing more than one guidewire to complete the maneuver, and will help to avoid needing a stiffer introducer to penetrate the femoral artery at the access site.

6. The degree of stiffness and flexibility of the guidewire can be precisely matched to the properties of the introducer and sheath to create a smoothly functioning system during each necessary maneuver.

The present invention can also be provided in a kit, where the kit comprises the sheath 50 and modified introducer 54, packaged with a correlating guidewire 66 specifically designed to expedite the functions of the sheath.

The above descriptions of the present invention are specific embodiments of the present invention and are not limited to the above descriptions and uses. Various sizes of sheaths and guidewires can be used, and can be made of various materials known to one of ordinary skill in the art. The flexibility and stiffness of the sheath, introducer and guidewire can also vary.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A method of providing vascular access, the method comprising:
inserting a guidewire having a flexible tip into an iliac artery and into an aorta;
inserting a sheath having an introducer therethrough, the introducer having an angulated flexible tip, into the iliac artery over the guidewire, wherein the angulated tip of the introducer is also inserted into the aorta; and
pulling back the guidewire from the introducer so that the angulated flexible tip of the introducer falls over an aortic bifurcation into a contralateral iliac artery.

2. The method of providing vascular access of claim 1, the method further comprising:
injecting dye through the sheath through sideholes on the flexible tip of the introducer to allow vessels to be visualized through x-rays.

3. The method of providing vascular access of claim 2, the method further comprising:
re-inserting the guidewire so that it travels through the sheath and introducer into the contralateral iliac artery.

4. The method of providing vascular access of claim 3, the method further comprising:
advancing the sheath and introducer over the guidewire over the aortic bifurcation and into the contralateral iliac artery.

5. The method of providing vascular access of claim 4, the method further comprising:
removing the guidewire and introducer from the sheath.

6. The method of providing vascular access of claim 1, wherein the flexible tip of the guidewire is approximately 10 cm in length and the guidewire is stiff proximate to the tip of the guidewire.

7. The method of providing vascular access of claim 6, wherein the introducer is tapered.

8. The method of providing vascular access of claim 6, wherein the diameter of the introducer and sheath is such that the introducer fits snugly into the sheath to provide a transitionless seam of the introducer from the sheath.

9. A device for vascular access, the device comprising:
a sheath having a distal end and a proximal end; and
an introducer running through the sheath and extending past a distal end of the sheath, the introducer having a flexible angulated tip at a distal end of the introducer;
wherein the introducer fits snugly into the sheath to provide a transitionless extension of the introducer from the sheath.

10. The device for vascular access of claim 9, the device further comprising:
a valve-like hub at a proximal end of the sheath.

11. The device for vascular access of claim 9, the device further comprising:
a stopcock and sidearm at a proximal end of the sheath for injecting fluid or dye through the sheath and for measuring pressure.

12. The device for vascular access of claim 9, the device further comprising:
a plurality of sideholes on the flexible angulated tip of the introducer.

13. The device for vascular access of claim 9, wherein the introducer is tapered, having a larger diameter at its proximal end and a smaller diameter at its tip.

14. The device for vascular access of claim 9, wherein the introducer is hydrophilic.

15. The device for vascular access of claim 9, wherein the flexibility of the transducer varies from more flexible at its tip to less flexible more proximally.

16. The device for vascular access of claim 9, wherein the introducer protrudes approximately 8-10 cm beyond the distal end of the sheath.

17. The device for vascular access of claim 9, wherein the introducer is made of a continuous metal or polymeric coil.

18. The device for vascular access of claim 9, wherein the sheath ranges in length between 45 cm to 90 cm.

19. A kit for vascular access, the kit comprising:
a sheath having a distal end and a proximal end;
an introducer running through the sheath and extending past a distal end of the sheath, the introducer having a flexible angulated tip at a distal end of the introducer; and
a guidewire running through the sheath and introducer; the guidewire having a flexible tip at a distal end.

20. The kit for vascular access of claim 19, wherein the guidewire is stiff proximal to the flexible tip.

21. The kit for vascular access of claim 20, wherein the stiffness of the wire correlates with a stiffness of the introducer.

22. The kit for vascular access of claim 19, wherein the flexible tip of the guidewire runs a length of approximately 10 cm.

23. The kit for vascular access of claim 19, wherein the introducer is tapered.

24. The kit for vascular access of claim 19, wherein the guidewire has an angulated flexible tip.
